(19) United States
(12) Patent Application Publication
(10) Pub. No.: US 2023/0096740 A1
(43) Pub. Date: Mar. 30, 2023

(54) Insertion Assemblies of Rapidly Insertable Central Catheters and Methods Thereof

(71) Applicant: Bard Access Systems, Inc., Salt Lake City, UT (US)

(72) Inventors: Justin Bechstein, Philadelphia, PA (US); Joe Spataro, Cottonwood Heights, UT (US); Carter Biviano, Philadelphia, PA (US); Sharon West, Elkins Park, PA (US); Kyle G. Thornley, Farmington, UT (US); Glade H. Howell, Draper, UT (US); Daniel B. Blanchard, Bountiful, UT (US); Christopher J. Murray, Philadelphia, PA (US); Eric W. Lindekugel, Salt Lake City, UT (US); Jenna Garafalo, San Jose, CA (US); David R. Schiff, Highland Park, NJ (US)

(21) Appl. No.: 17/953,959

(22) Filed: Sep. 27, 2022

Related U.S. Application Data
(60) Provisional application No. 63/322,056, filed on Mar. 21, 2022.
(60) Provisional application No. 63/271,043, filed on Oct. 22, 2021.
(60) Provisional application No. 63/249,009, filed on Sep. 27, 2021.

(51) Int. Cl. A61M 25/01; A61M 25/00

(52) U.S. Cl. CPC A61M 25/01; A61M 25/0097

(57) ABSTRACT

Disclosed are insertion assemblies of rapidly insertable central catheters (“RICCs”) and methods thereof. For example, a RICC insertion assembly can include a RICC, an introducer needle, an access guidewire, and a coupler assembly coupling the foregoing together. The introducer needle can include a needle hub over both a sheath and a needle shaft. The sheath can seal a needle slot except for that under a sheath opening. A distal end of the access guidewire can be disposed in the introducer needle just proximal of a needle tip. The coupler assembly can include a nose piece and a tail piece coupled together. The tail piece can include an extension arm by which a proximal end of the access guidewire is held. The access guidewire can enforce a loop in the access guidewire over which the RICC is disposed, thereby keeping the RICC insertion assembly in a relatively compact form.

### PRIORITY

This application claims the benefit of priority to U.S. Provisional Application No. 63/249,009, filed Sep. 27, 2021; U.S. Provisional Application No. 63/271,043, filed Oct. 22, 2021; and U.S. Provisional Application No. 63/322,056, filed Mar. 21, 2022, each of which is incorporated by reference in its entirety into this application.

### BACKGROUND

Central venous catheter (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are insertion assemblies of rapidly insertable central catheters (“RICCs”) and methods thereof that address the foregoing.

### SUMMARY

Disclosed herein is a RICC insertion assembly including, in some embodiments, a RICC, an introducer needle, an access guidewire, and a coupler assembly coupling the RICC, the introducer needle, and the access guidewire together. The introducer needle includes a needle shaft, a sheath over the needle shaft, and a needle hub over proximal portions the needle shaft and the sheath. The needle shaft includes a longitudinal needle slot extending from a proximal portion of the needle shaft through a distal needle tip. The sheath seals the needle slot thereunder except for the needle slot under a sheath opening in a proximal portion of the sheath. The access guidewire includes a proximal portion including a proximal end and a distal portion including a distal end. The distal end of the access guidewire is disposed in the introducer needle just proximal of the needle tip in at least a ready-to-deploy state of the RICC insertion assembly. The coupler assembly includes a nose piece and a tail piece coupled to the nose piece. The tail piece of the coupler assembly includes an extension arm by which the proximal end of the access guidewire is held. The proximal and distal ends of the access guidewire enforce a loop in the access guidewire over which the RICC is disposed in at least the ready-to-deploy state of the RICC insertion assembly.

In some embodiments, the nose piece of the coupler assembly includes a nose-piece housing. The nose-piece housing defines a valve-module compartment and a lock-ring compartment proximal of the valve-module compartment.

In some embodiments, the nose piece of the coupler assembly further includes a valve module disposed in the valve-module compartment. The valve module includes an introducer-needle passageway and an access-guidewire passageway connecting thereto. Within the introducer-needle passageway and the access-guidewire passageway, the introducer needle and the distal portion of the access guidewire are correspondingly sealed in at least the ready-to-deploy state of the RICC insertion assembly. Sealing the introducer needle and the access guidewire in the valve module enables leak-free aspiration through the introducer needle.

In some embodiments, the sheath opening of the sheath opens toward an opposite side of the coupler assembly than that including the extension arm. The access-guidewire passageway connects to the sheath opening in at least the ready-to-deploy state of the RICC insertion assembly with the introducer needle within the introducer-needle passageway.

In some embodiments, the valve module includes an integrated blade disposed in the needle slot under a distal end of the sheath opening. The blade includes a distal-facing blade edge configured to cut the sheath off the needle shaft during withdrawal of the introducer needle from the coupler assembly by the needle hub. Cutting the sheath off the needle shaft allows the access guidewire to escape from the needle shaft by way of the needle slot thereof.

In some embodiments, the nose piece of the coupler assembly further includes a lock ring captively but rotatably disposed in the lock-ring compartment.

In some embodiments, the tail piece of the coupler assembly includes an introducer-needle cradle distally extending from the tail piece. The introducer-needle cradle is configured to cradle a needle-hub extension tube distally extending from the needle hub in at least a ready-to-deploy state of the RICC insertion assembly.

In some embodiments, a wall of the introducer-needle cradle approximates a longitudinal cross-section of a pipe. In addition, the needle-hub extension tube includes an arcuate extension-tube protrusion in a distal portion thereof configured to at least partially complement the wall of the introducer-needle cradle. Together, the wall of the introducer-needle cradle and the extension-tube protrusion form a two-piece axle configured for rotating the lock ring thereupon.

In some embodiments, the tail piece of the coupler assembly includes a longitudinal tail-piece slot to which the introducer-needle cradle opens. The tail-piece slot is configured to allow the access guidewire to escape from the tail piece subsequent to both withdrawal of the introducer needle from the coupler assembly and separation of the tail piece from the coupler assembly.

In some embodiments, the lock ring includes a lock-ring protrusion protruding toward a centerline of the lock ring. The lock-ring protrusion is positioned on the lock ring to create a tortured guidewire passageway therearound for restraining the access guidewire from moving when a lock-ring tab extending away from the centerline of the lock ring is to a side of a tab slot of the nose-piece housing through which tab slot the lock-ring tab extends.

In some embodiments, the lock-ring protrusion is further positioned on the lock ring to create an open guidewire passageway for the access guidewire when the lock-ring tab is to an opposite side of the tab slot of the nose-piece housing of the coupler assembly.

In some embodiments, the lock ring includes a lock-ring gap on an opposite side of the lock ring from the lock-ring protrusion. The lock-ring gap is configured to allow the access guidewire to escape from the lock ring during separation of the nose piece from the access guidewire.

In some embodiments, the nose-piece housing of the nose piece includes a longitudinal nose piece-housing slot on a same side of the coupler assembly as the extension arm of the tail piece. The nose piece-housing slot is configured to allow the access guidewire to escape from the nose piece during separation of the nose piece from the access guidewire.

In some embodiments, the needle hub of the introducer needle includes a pair of clip arms distally extending from opposite sides of the needle hub. The clip arms are configured to extend completely over corresponding sides of the tail piece of the coupler assembly to at least the nose piece thereof in at least a ready-to-deploy state of the RICC insertion assembly.

In some embodiments, the clip arms include textured outer clip-arm surfaces and the nose piece of the coupler assembly includes textured outer nose-piece surfaces on sides of the nose piece corresponding with the clip arms. The outer clip-arm surfaces and the outer nose-piece surfaces are configured to form a pair of textured grip pads on opposite sides of the RICC insertion assembly across the nose piece and the needle hub.

In some embodiments, the extension arm terminates with an extension-arm connector connected to a Luer connector of the RICC in at least the ready-to-deploy state of the RICC insertion assembly.

In some embodiments, the extension-arm connector includes a male Luer connector. The Luer connector of the RICC is a female counterpart to the male Luer connector of the extension-arm connector.

In some embodiments, the RICC insertion assembly further includes a syringe fluidly coupled to the introducer needle in at least the ready-to-deploy state of the RICC insertion assembly.

Also disclosed herein is a method for inserting a RICC into a blood-vessel lumen of a patient. The method includes a RICC insertion assembly-obtaining step, a needle tract-establishing step, an access guidewire-advancing step, an introducer needle-withdrawing step, a tail piece-separating step, and a RICC-advancing step. The RICC insertion assembly-obtaining step includes obtaining a RICC insertion assembly. The RICC insertion assembly includes the RICC, an introducer needle including a sheath over a needle shaft, and an access guidewire coupled together by a coupler assembly. A proximal end of the access guidewire is held by an extension arm of a tail piece of the coupler assembly. In addition, a distal end of the access guidewire is disposed in the introducer needle by way of a valve module disposed in a nose-piece housing of a nose piece of the coupler assembly such that the proximal and distal ends of the access guidewire enforce a loop in the access guidewire. The RICC is disposed over the access guidewire in at least a ready-to-deploy state of the RICC insertion assembly. The needle tract-establishing step includes establishing a needle tract from an area of skin to the blood-vessel lumen with the introducer needle. The access guidewire-advancing step includes advancing the distal end of the access guidewire into the blood-vessel lumen from its initial location in the needle shaft just proximal of a needle tip of the needle shaft. The introducer needle-withdrawing step includes withdrawing the introducer needle by a needle hub thereof from the coupler assembly leaving the access guidewire in place in the blood-vessel lumen. The needle shaft includes a longitudinal needle slot extending from a proximal portion of the needle shaft through the needle tip that allows the access guidewire to escape from the needle shaft with the withdrawing of the introducer needle from the coupler. The tail piece-separating step includes separating the tail piece from the nose piece of the coupler assembly. The tail piece includes a longitudinal tail-piece slot that allows the access guidewire to escape from the tail piece subsequent to the withdrawal of the introducer needle from the coupler assembly. The RICC-advancing step includes advancing a catheter tube of the RICC over the access guidewire for the inserting of the RICC into the blood-vessel lumen.

In some embodiments, the method further includes a blood-aspirating step. The blood-aspirating step includes aspirating blood with a syringe coupled to the needle hub of the introducer needle for confirmation the needle tract extends into the blood-vessel lumen before the withdrawing of the introducer needle from the coupler assembly. The sheath is over the needle shaft sealing the needle slot under the sheath for the aspirating of the blood with the syringe.

In some embodiments, the withdrawing of the introducer needle from the coupler assembly includes simultaneously cutting the sheath off the needle shaft with an integrated blade of a valve module disposed in a valve-module compartment defined by the nose-piece housing of the nose piece. The cutting of the sheath off the needle shaft allows the access guidewire to escape from the needle shaft by way of the needle slot thereof.

In some embodiments, the method further includes an access guidewire-restraining step. The access guidewire-restraining step includes restraining the access guidewire from moving in the RICC insertion assembly after the advancing of the access guidewire into the blood-vessel lumen so as to not withdraw the access guidewire from the blood-vessel lumen during the withdrawing of the introducer needle from the coupler assembly or the separating of the tail piece from the nose piece of the coupler assembly. The restraining of the access guidewire from moving in the RICC insertion assembly includes rotating a lock ring having a lock-ring protrusion that protrudes toward a centerline of the lock ring. The rotating of the lock ring in the access guidewire-restraining step creates a tortured guidewire passageway around the lock-ring protrusion for the restraining of the access guidewire from moving in the RICC insertion assembly.

In some embodiments, the rotating of the lock ring includes pushing a lock-ring tab of the lock ring to a side or an opposite side of a tab slot of the nose-piece housing through which tab slot the lock-ring tab extends.

In some embodiments, the rotating of the lock ring is over a two-piece axle formed between a wall of an introducer-needle cradle and an arcuate extension-tube protrusion of a needle-hub extension tube. The introducer-needle cradle distally extends from the tail piece of the coupler assembly, and the needle-hub extension tube distally extends from the needle hub of the introducer needle. The rotating of the lock ring is also within a lock-ring compartment defined by the nose-piece housing of the nose piece in which the lock ring is captively disposed.

In some embodiments, the method further includes an access guidewire-releasing step. The access guidewire-releasing step includes releasing the access guidewire to move in the nose piece after the withdrawing of the introducer needle from the coupler assembly and the separating of the tail piece from the nose piece of the coupler assembly. The releasing of the access guidewire to move in the nose piece includes rotating the lock ring opposite to that for the restraining of the access guidewire from moving in the RICC insertion assembly. The rotating of the lock ring in the access guidewire-releasing step creates an open guidewire passageway for the releasing of the access guidewire to move in the nose piece.

In some embodiments, the method further includes a nose piece-separating step. The nose piece-separating step includes separating the nose piece from the access guidewire after the releasing of the access guidewire to move in the nose piece. The lock ring includes a lock-ring gap on an opposite side of the lock ring from the lock-ring protrusion. The lock-ring gap allows the access guidewire to escape from the lock ring during the separating of the nose piece from the access guidewire.

In some embodiments, the nose-piece housing of the nose piece includes a longitudinal nose piece-housing slot on a same side of the coupler assembly as the extension arm of the tail piece of the coupler assembly. The nose piece-housing slot allows the access guidewire to escape from the nose piece during the separating of the nose piece from the access guidewire.

In some embodiments, the tail piece becomes a handle for the access guidewire after the separating of the tail piece from the nose piece of the coupler assembly.

In some embodiments, the method further includes an access guidewire-withdrawing step. The access guidewire-withdrawing step includes withdrawing the access guidewire from the blood-vessel lumen after the inserting of the RICC into the blood-vessel lumen. The withdrawing of the access guidewire from the blood-vessel lumen also includes removing a Luer connector of the RICC from an extension-arm connector of the extension arm, thereby decoupling the RICC from the tail piece.

These and other features of the concepts provided herein will become more apparent to those of skill in the art in view of the accompanying drawings and following description, which describe particular embodiments of such concepts in greater detail.

### DRAWINGS

FIG. 1 illustrates a side view of a RICC insertion assembly in accordance with some embodiments.

FIG. 2 illustrates a side view of an introducer-needle assembly including an introducer needle coupled with a coupler assembly in accordance with some embodiments.

FIG. 3 illustrates a top view of the introducer-needle assembly in accordance with some embodiments.

FIG. 4 illustrates a bottom view of the introducer-needle assembly in accordance with some embodiments.

FIG. 5 illustrates a side view of the introducer-needle assembly with the introducer needle being withdrawn from the coupler assembly in accordance with some embodiments.

FIG. 6 illustrates an exploded view of the introducer-needle assembly without a needle shaft or a sheath of the introducer needle.

FIG. 7 illustrates an exploded view of the coupler assembly with an access guidewire in an open guidewire passageway therethrough in accordance with some embodiments.

FIG. 8 illustrates an exploded view of the coupler assembly with the access guidewire in a tortured guidewire passageway therethrough in accordance with some embodiments.

FIG. 9 illustrates a longitudinal cross section of the introducer-needle assembly without a nose-piece housing of a nose piece of the coupler assembly.

FIG. 10 illustrates a lock ring on a two-piece axle from a proximal end of the introducer-needle assembly without the nose-piece housing of the nose piece, a valve module of the nose piece, or the needle shaft or the sheath thereover, a lock-ring protrusion of the lock ring creating the tortured guidewire passageway in accordance with some embodiments.

FIG. 11 illustrates the lock-ring protrusion of the lock ring creating the open guidewire passageway in accordance with some embodiments.

FIG. 12 illustrates a longitudinal cross section of the nose piece of the coupler assembly in accordance with some embodiments.

FIG. 13 illustrates a side view of the introducer needle in accordance with some embodiments.

FIG. 14 illustrates a top view of the introducer needle in accordance with some embodiments.

FIG. 15 illustrates a top view of the sheath over the needle shaft as in the introducer needle in accordance with some embodiments.

FIG. 16 illustrates a top view of the sheath in accordance with some embodiments.

FIG. 17 illustrates a top view of the needle shaft in accordance with some embodiments.

FIG. 18 illustrates a RICC of the RICC insertion assembly in accordance with some embodiments.

FIG. 19 illustrates a detailed view of a distal portion of a catheter tube of the RICC in accordance with some embodiments.

FIG. 20 illustrates a transverse cross section of the distal portion of the catheter tube in accordance with some embodiments.

FIG. 21 illustrates another transverse cross section of the distal portion of the catheter tube in accordance with some embodiments.

FIG. 22 illustrates a longitudinal cross section of the distal portion of the catheter tube in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above with respect to the Seldinger technique, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are insertion assemblies of RICCs and methods thereof. For example, a RICC insertion assembly can include a RICC, an introducer needle, an access guidewire, and a coupler assembly coupling the foregoing together. The introducer needle can include a needle hub over both a sheath and a needle shaft. The sheath can seal a needle slot except for that under a sheath opening. A distal end of the access guidewire can be disposed in the introducer needle just proximal of a needle tip. The coupler assembly can include a nose piece and a tail piece coupled together. The tail piece can include an extension arm by which a proximal end of the access guidewire is held. The access guidewire can enforce a loop in the access guidewire over which the RICC is disposed, thereby keeping the RICC insertion assembly in a relatively compact form.

The foregoing features as well as other features of the RICC insertion assemblies and subassemblies thereof (e.g., coupler assemblies, introducer-needle assemblies, etc.) will become more apparent to those of skill in the art in view of the accompanying drawings and following description, which describe particular embodiments of the RICC insertion assemblies. While the RICC insertion assemblies include central catheters, it should be understood that various catheters can be incorporated into catheter insertion assemblies like the RICC insertion assemblies provided herein. Indeed, peripherally inserted central catheters (“PICCs”), dialysis catheters, or the like can also be incorporated into catheter insertion assemblies.

### RICC Insertion Assemblies

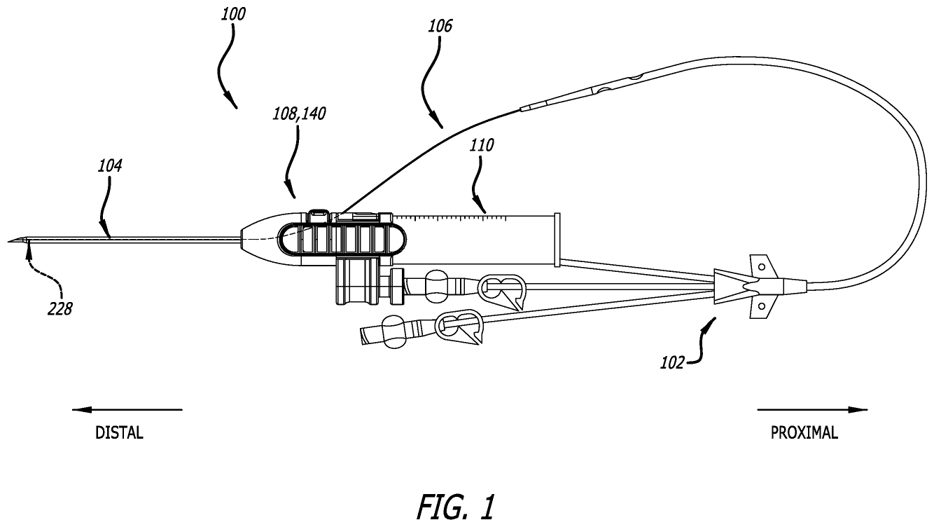

FIG. 1 illustrates a RICC insertion assembly 100 in accordance with some embodiments.

As shown, the RICC insertion assembly 100 includes a RICC 102, an introducer needle 104, an access guidewire 106, and a coupler assembly 108 coupling the RICC 102, the introducer needle 104, and the access guidewire 106 together in at least a ready-to-deploy state of the RICC insertion assembly 100. Notably, the proximal end of the access guidewire 106 is held by the extension arm 214 of the tail piece 180 of the coupler assembly 108. In addition, the distal end of the access guidewire 106 is disposed in the needle lumen 156 of the introducer needle 104 just proximal of the needle tip 148 in at least the ready-to-deploy state of the RICC insertion assembly 100. The proximal and distal ends of the access guidewire 106 thereby enforce a loop in the access guidewire 106. The RICC 102 is disposed over the loop in at least the ready-to-deploy state of the RICC insertion assembly 100 keeping the RICC insertion assembly 100 in a relatively compact form.

The RICC insertion assembly 100 can further include a syringe 110 fluidly coupled to the introducer needle 104 in at least the ready-to-deploy state of the RICC insertion assembly 100. As set forth below, the sheath 144 seals the needle slot 150 of the needle shaft 142. In particular, the sheath 144 seals the needle slot 150 outside of the valve module 186. The valve module 186, in turn, seals over the sheath opening 160 of the sheath 144 that opens to the needle slot 150. The valve module 186 also seals around the access guidewire 106. Such seals enable leak-free blood aspiration through the introducer needle 104 with the syringe 110 in accordance with the blood-aspirating step of the method set forth below.

Lastly, any component of the RICC insertion assembly 100 selected from at least the RICC 102, the introducer needle 104, the access guidewire 106, the coupler assembly 108, and the syringe 110, or any portion of the component selected from the foregoing components, can include an antimicrobial thereon or therein. In an example, the catheter tube 112 of the RICC 102 can include an antimicrobial coating on an abluminal surface of the catheter tube 112, a luminal surface of the catheter tube 112, or both. In another example, a pre-extrusion material of the catheter tube 112 can include the antimicrobial admixed therein such that the antimicrobial is incorporated into the catheter tube 112 when extruded, the antimicrobial protecting both the abluminal surface of the catheter tube 112 and the luminal surface of the catheter tube 112 from microbial contamination.

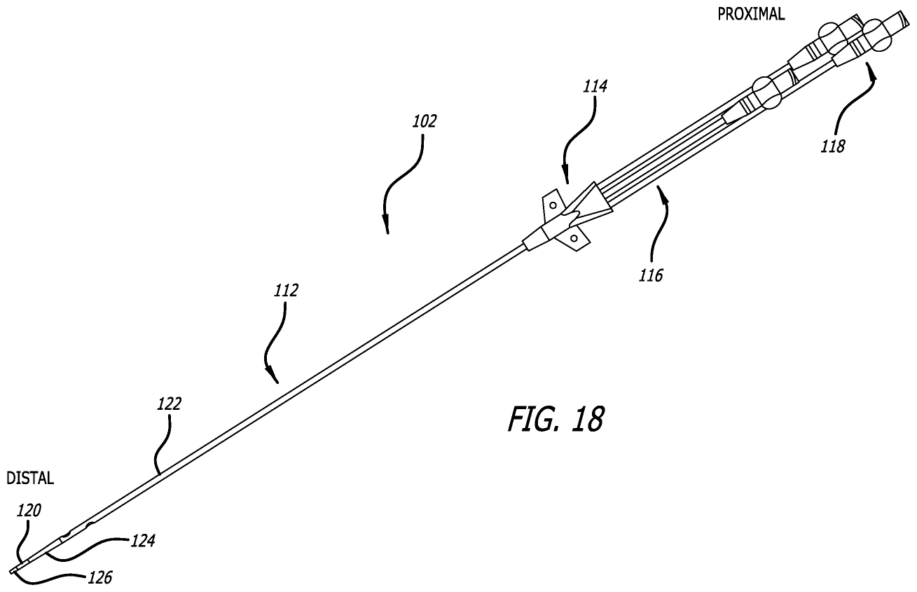

FIG. 18 illustrates the RICC 102 of the RICC insertion assembly 100 in accordance with some embodiments.

As shown, the RICC 102 includes a catheter tube 112, a catheter hub 114, one or more extension legs 116, and one or more extension-leg connectors 118.

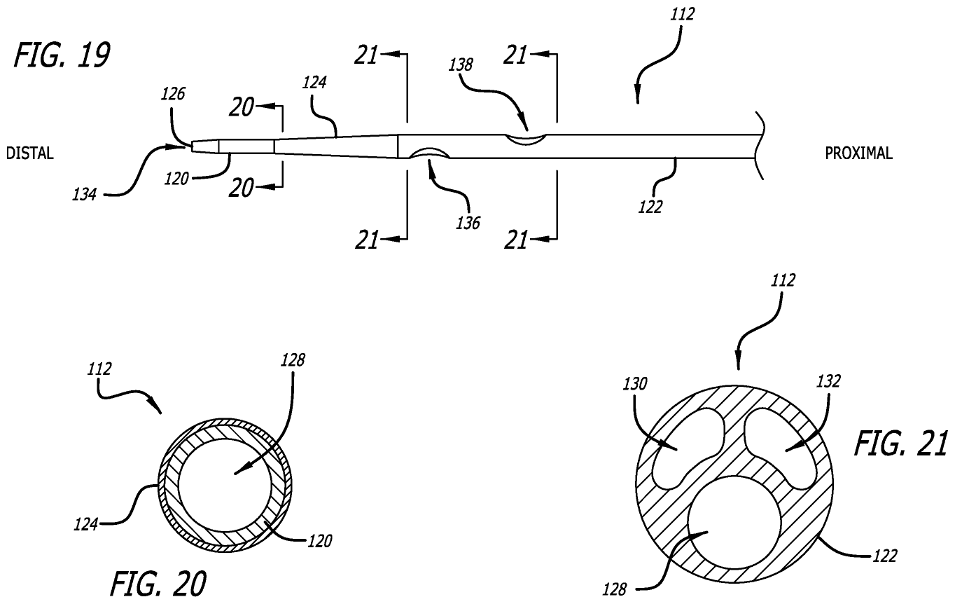

FIGS. 19-22 illustrate various views of the catheter tube 112 of the RICC 102 in accordance with some embodiments.

The catheter tube 112 includes a first section 120 in a distal portion of the catheter tube 112, a second section 122 in the distal portion of the catheter tube 112 proximal of the first section 120, and a tapered junction 124 between the first and second sections 120 and 122 of the catheter tube 112.

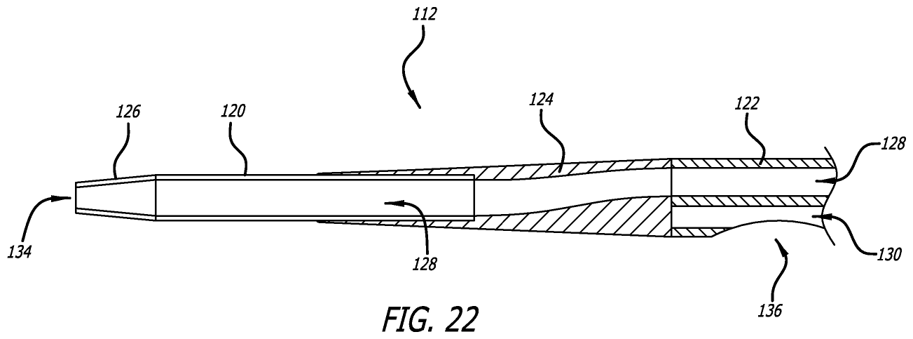

The first section 120 of the catheter tube 112 includes a catheter tip 126 having a relatively short taper from an outer diameter of a distal portion of the first section 120 distal of the junction 124 to an outer diameter of a distal end of the first section 120. The taper of the catheter tip 126 is configured for immediate dilation of tissue about a needle tract established with the introducer needle 104 up to the outer diameter of the distal portion of the first section 120 of the catheter tube 112. As best shown in FIG. 22, the first section 120 of the catheter tube 112 also includes a proximal portion disposed in a bore of a distal portion of the junction 124 and fixedly coupled thereto such as by a solvent bond, an adhesive bond, or a heat weld.

The second section 122 of the catheter tube 112 includes a consistent outer diameter over its length from a distal end of the second section 122 to a proximal end of the second section 122. The consistent diameter of the second section 122 of the catheter tube 112 is configured for smooth insertion into the needle tract and targeted vasculature subsequent to any dilation by the first section 120 of the catheter tube 112 and the junction 124. The distal end of the second section 122 of the catheter tube 112 has a flat face flush with the flat-faced proximal end of the junction 124 and fixedly coupled thereto such as by a solvent bond, an adhesive bond, or a heat weld.

The junction 124 includes a taper over its length from a proximal end of the junction 124 to a distal end of the junction 124. The taper of the junction 124 is configured for immediate dilation of the tissue about the needle tract from the outer diameter of the proximal portion of the first section 120 of the catheter tube 112 to the outer diameter of the second section 122 of the catheter tube 112. An abluminal surface of the junction 124 smoothly transitions from an abluminal surface of the first section 120 of the catheter tube 112 to an abluminal surface of the second section 122 of the catheter tube 112 without edges that catch on skin when the catheter tube 112 is inserted into the needle tract. In addition to the edges being minimal to negligible, the edges can include solvent-interdiffused polymeric material of the polymeric materials from which the catheter tube 112 is formed, which smoothens the transitions from the first section 120 of the catheter tube 112 to the junction 124 and from the junction 124 to the second section 122 of the catheter tube 112. Notably, the junction 124 has a length approximately commensurate with a length of an exposed portion of the first section 120 of the catheter tube 112 or between lengths of exposed portions of the first and second sections 120 and 122 of the catheter tube 112. As such, the length of the exposed portion of the first section 120 of the catheter tube 112 is less than the length of the junction 124 up to approximately commensurate with the length of the junction 124.

The first section 120 of the catheter tube 112 is formed of a first polymeric material (e.g., a polytetrafluoroethylene, a polypropylene, or a polyurethane) having a first durometer. The second section 122 of the catheter tube 112 is formed of a second polymeric material (e.g., a polyvinyl chloride, a polyethylene, another polyurethane, or a silicone) having a second durometer less than the first durometer. For example, the first section 120 of the catheter tube 112 can be formed of a first polyurethane having the first durometer while the second section 122 of the catheter tube 112 can be formed of a second, different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having the second durometer less than the first durometer. Indeed, polyurethanes are advantageous for the catheter tube 112 in that polyurethanes can be relatively rigid at room-temperature but become more flexible in vivo at body temperature, which reduces irritation to vessel walls as well as phlebitis. Polyurethanes are also advantageous in that they can be less thrombogenic than some other polymers. The junction 124 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 120 of the catheter tube 112, the second section 122 of the catheter tube 112, and the junction 124 between the first and second sections 120 and 122 of the catheter tube 112 set forth above, the catheter tube 112 possesses a column strength sufficient to prevent buckling of the catheter tube 112 when inserted into a needle tract established by the introducer needle 104. The column strength of the catheter tube 112 is also sufficient to prevent buckling of the catheter tube 112 when advanced through a vasculature of a patient without dilation of tissue about the needle tract or any blood vessels of the vasculature beforehand with a separate dilator.

The catheter tube 112 includes one or more catheter-tube lumens extending through the catheter tube 112; however, only one catheter-tube lumen typically extends from a proximal end of the catheter tube 112 to a distal end of the catheter tube 112 in a multiluminal RICC (e.g., a diluminal RICC, a triluminal RICC, a tetraluminal RICC, a pentaluminal RICC, a hexaluminal RICC, etc.). (See FIGS. 19-21.) Indeed, the first section 120 of the catheter tube 112 typically includes a single lumen therethrough as shown in FIGS. 20 and 21.

The catheter hub 114 is coupled to a proximal portion of the catheter tube 112. The catheter hub 114 includes one or more catheter-hub lumens corresponding in number to the one-or-more catheter-tube lumens. The one-or-more catheter-hub lumens extend through an entirety of the catheter hub 114 from a proximal end of the catheter hub 114 to a distal end of the catheter hub 114.

Each extension leg of the one-or-more extension legs 116 is coupled to the catheter hub 114 by a distal portion thereof. The one-or-more extension legs 116 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension-leg connector of the one-or-more extension-leg connectors 118 is over a proximal portion of an extension leg of the one-or-more extension legs 116. For example, each extension-leg connector of the one-or-more extension-leg connectors 118 can be a Luer connector (e.g., a female Luer connector) over a proximal portion of an extension leg of the one-or-more extension legs 116. Through such an extension-leg connector, a corresponding extension leg and the extension-leg lumen thereof can be connected to another medical device and a lumen thereof. However, in at least the ready-to-deploy state of the RICC insertion assembly 100 at least one extension-leg connector (e.g., the extension-leg connector including part of the primary lumen 128 of the RICC 102) is connected to the extension-arm connector 220 of the extension arm 214 of the tail piece 180 to enforce the loop in the access guidewire 106 and the RICC 102 thereover.

As shown, the RICC 102 is a triluminal RICC including a set of three lumens; however, the RICC 102 is not limited to the set of the three lumens as set forth above. The set of three lumens includes a primary lumen 128, a secondary lumen 130, and a tertiary lumen 132 formed of fluidly connected portions of three catheter-tube lumens, three catheter-hub lumens, and three extension-leg lumens. The primary lumen 128 has a primary-lumen aperture 134 in the distal end of the first section 120 of the catheter tube 112, which corresponds to the distal end of the catheter tube 112 and a distal end of the RICC 102. The secondary lumen 130 has a secondary-lumen aperture 136 in a side of the distal portion of the catheter tube 112. The tertiary lumen 132 has a tertiary-lumen aperture 138 in the side of the distal portion of the catheter tube 112 proximal of the secondary-lumen aperture 136.

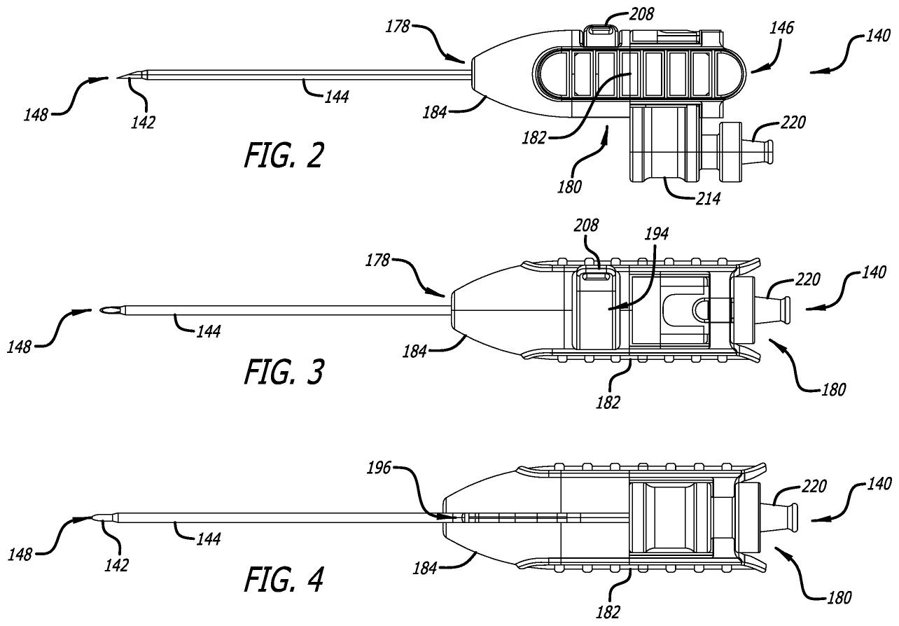

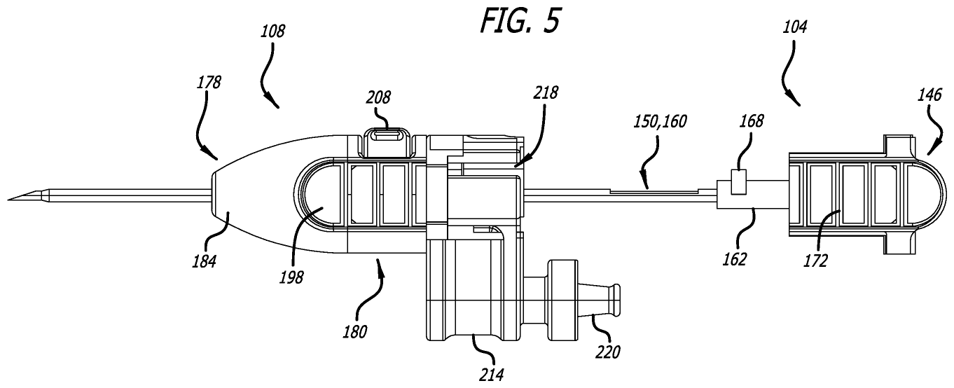

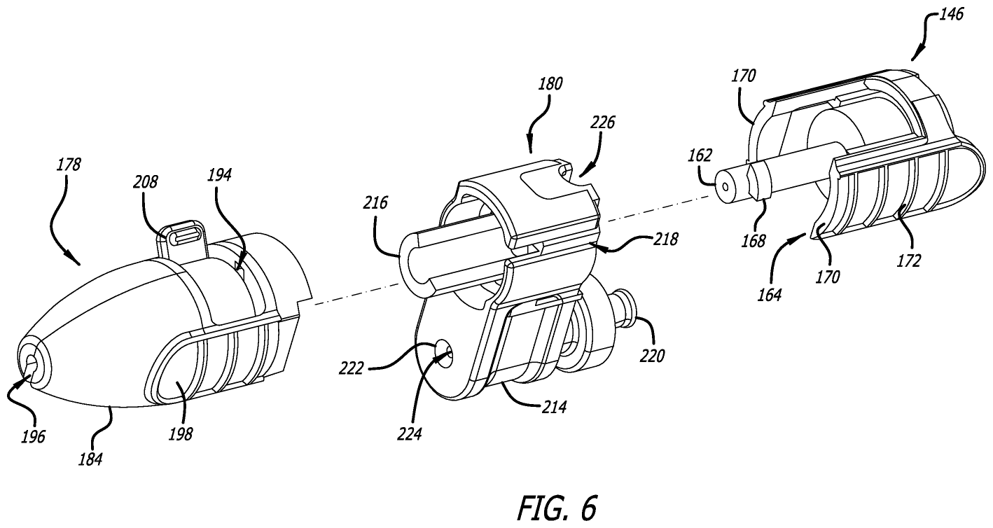

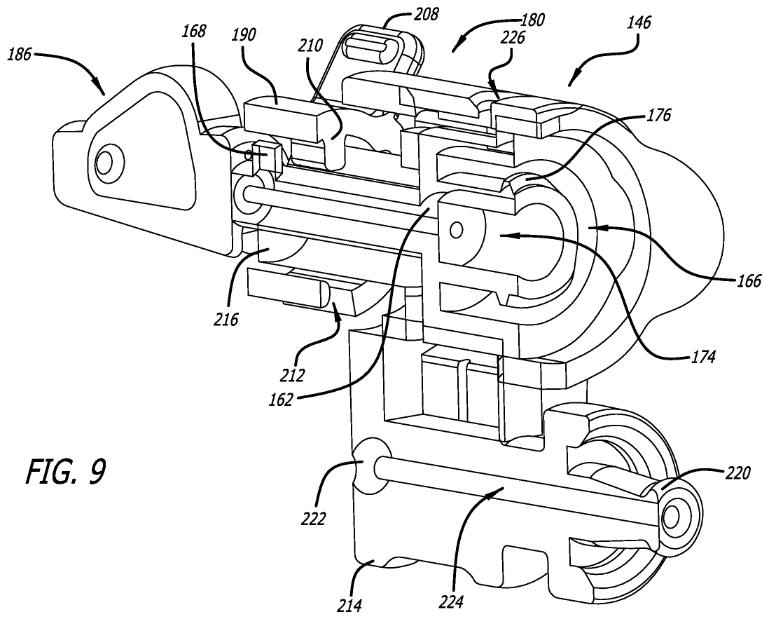

FIGS. 2-4 illustrate various views of an introducer-needle assembly 140 including the introducer needle 104 coupled with the coupler assembly 108 in accordance with some embodiments. FIG. 5 illustrates the introducer-needle assembly 140 with the introducer needle 104 being withdrawn therefrom in accordance with some embodiments. And for exposition only, FIG. 6 illustrates an exploded view of the introducer-needle assembly 140 without the needle shaft 142 or the sheath 144 of the introducer needle 104, and FIG. 9 illustrates a longitudinal cross section of the introducer-needle assembly 140 without the nose-piece housing 184 of the nose piece 178 of the coupler.

As shown, the introducer-needle assembly 140 is a subassembly of the RICC insertion assembly 100. Indeed, the introducer-needle assembly 140 includes the introducer needle 104 and the coupler assembly 108 coupled together. The coupler assembly 108 is, in turn, a subassembly of each of the RICC insertion assembly 100 and the introducer-needle assembly 140. As set forth in more detail below, the coupler assembly 108 includes the nose piece 178 and the tail piece 180 thereof coupled together.

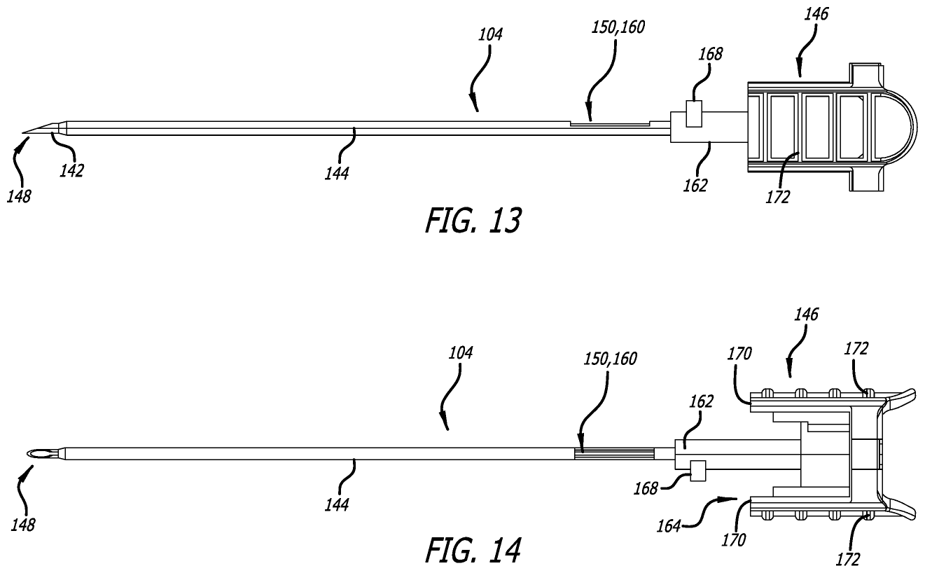

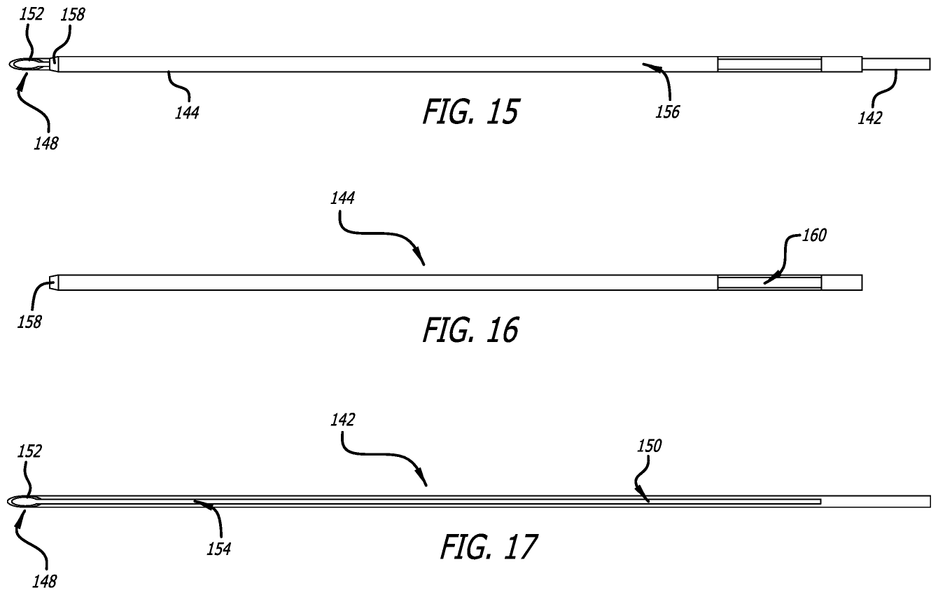

FIGS. 13-17 illustrate various views of the introducer needle 104 or components thereof in accordance with some embodiments.

As shown, the introducer needle 104 includes a needle shaft 142, a sheath 144 over the needle shaft 142, and a needle hub 146 over both a proximal portion of the needle shaft 142 and a proximal portion of the sheath 144. In at least the ready-to-deploy state of the RICC insertion assembly 100, the needle shaft 142 and the sheath 144 extend from the needle hub 146, through the valve module 186, and out a distal end of the nose piece 178 of the coupler assembly 108.

The needle shaft 142 includes a needle tip 148 in a distal portion of the needle shaft 142 and a longitudinal needle slot 150 (i.e., a missing portion of a wall of the needle shaft 142) extending from at least the proximal portion of the needle shaft 142 through the needle tip 148.

The needle tip 148 includes a bevel 152 having a tip bevel and a primary bevel proximal of the tip bevel. While not shown, a tip-bevel angle of the tip bevel is greater than a primary-bevel angle of the primary bevel such that the bevel 152 provides a smooth transition over the needle tip 148. Such a needle tip is thusly configured for establishing a needle tract from an area of skin into a blood-vessel lumen of a patient in accordance with the needle tract-establishing step of the method set forth below.

The needle slot 150 extends from at least the proximal portion of the needle shaft 142 through the needle tip 148, thereby forming a needle channel 154 along at least a majority of a length of the needle shaft 142; however, the needle slot 150 can extend through both the proximal end of the needle shaft 142 and the needle tip 148, thereby forming the needle channel 154 along an entirety of the length of the needle shaft 142. As the needle slot 150 is the missing portion of the wall of the needle shaft 142, the needle slot 150 has a defined width, which width is sized in accordance with an outer diameter of the access guidewire 106. Such a width allows the access guidewire 106 to pass from the proximal portion of the needle shaft 142 through the needle tip 148 when the introducer needle-withdrawing step of the method set forth below is performed.

While the needle shaft 142 includes the needle channel 154 due to the needle slot 150, it should be understood the introducer needle 104 includes a needle lumen 156. The needle lumen 156 results from the combination of the needle shaft 142 and the sheath 144 over the needle shaft 142. Indeed, the sheath 144 over the needle shaft 142 seals the needle slot 150 thereunder forming the needle lumen 156 of the introducer needle 104 from the needle channel 154 of the needle shaft 142, thereby enabling leak-free blood aspiration through the introducer needle 104 with the syringe 110 in accordance with the blood-aspirating step of the method set forth below.

The sheath 144 includes a sheath tip 158 in a distal portion of the sheath 144 and a sheath opening 160 in a side of the proximal portion of the sheath 144.

The sheath tip 158 includes a relatively short taper from an outer diameter of the distal portion of the sheath 144 to an outer diameter of a distal end of the sheath 144, the latter of which is commensurate with an outer diameter of the distal portion of the needle shaft 142. The taper has a taper angle less than the primary-bevel angle of the primary bevel of the needle tip 148, which, in turn, is less than the tip-bevel angle of the tip bevel of the needle tip 148. The sheath tip 158 having such a taper is configured to provide a smooth transition from the needle tip 148 to the sheath body for the needle tract-establishing step of the method set forth below.

The sheath opening 160 opens to the needle slot 150 of the needle shaft 142 allowing the access guidewire 106 to pass through the sheath opening 160 and into the needle channel 154 or the needle lumen 156 formed therefrom in at least the ready-to-deploy state of the RICC insertion assembly 100. Notably, the sheath opening 160 opens toward an opposite side of the coupler assembly 108 than that including the extension arm 214 in at least the ready-to-deploy state of the RICC insertion assembly 100. The sheath opening 160 has a width approximately commensurate with a width of the needle slot 150, which, in turn, is sized in accordance with the diameter of the access guidewire 106. The sheath opening 160 also has a length sufficient to allow the access guidewire 106 to pass through the sheath opening 160 and into the needle slot 150 or the needle lumen 156 formed therefrom while also accommodating the blade 204 of the valve module 186 under a distal end of the sheath opening 160. Notably, the sheath 144 over the needle shaft 142 seals the needle slot 150 thereunder except for the needle slot 150 under the sheath opening 160. However, the valve module 186 seals over the needle slot 150 exposed by the sheath opening 160 by sealing the proximal portions of the needle shaft 142 and the sheath 144 therein, thereby enabling leak-free blood aspiration through the introducer needle 104 with the syringe 110 in accordance with the blood-aspirating step of the method set forth below.

The sheath 144, or a sheath body thereof, is formed of a polymeric material configured to facilitate a smooth, consistent insertion of the introducer needle 104 from an area of skin to a blood-vessel lumen of a patient in accordance with the needle tract-establishing step of the method set forth below. In addition, the polymeric material has mechanical properties at a thickness of the sheath 144 sufficient to withstand collapse of the sheath 144 into the needle slot 150 of the needle shaft 142 when the blood-aspirating step of the method set forth below is performed, notably, while also facilitating the cutting of the sheath 144 off the needle shaft 142 in accordance with the introducer needle-withdrawing step of the method set forth below. Such a polymeric material can include, but is not limited to, polyethylene, polypropylene, or polytetrafluoroethylene.

The needle hub 146 includes a needle-hub extension tube 162 distally extending from the needle hub 146, a needle-hub clip 164 distally extending over a portion of the needle-hub extension tube 162, and a needle-hub connector 166 in a proximal portion of the needle hub 146.

The needle-hub extension tube 162 distally extends from the needle hub 146 over the proximal portions of the needle shaft 142 and the sheath 144. Notably, the needle-hub extension tube 162 includes an arcuate extension-tube protrusion 168 in a distal portion thereof configured to at least partially complement the wall of the introducer-needle cradle 216 of the tail piece 180. Indeed, the needle-hub extension tube 162 extends into the lock-ring compartment 192 of the nose-piece housing 184 of the nose piece 178. In the lock-ring compartment 192, the extension-tube protrusion 168 forms a two-piece axle with the wall of the introducer-needle cradle 216 configured for rotating the lock ring 190 thereupon.

The needle-hub clip 164 includes a pair of clip arms 170 distally extending from opposite sides of the needle hub 146 over the needle-hub extension tube 162. The clip arms 170 are configured to extend completely over corresponding sides of the tail piece 180 of the coupler assembly 108 to at least the nose piece 178 of the coupler assembly 108 in at least a ready-to-deploy state of the RICC insertion assembly 100. Advantageously, the clip arms 170 can include textured outer clip-arm surfaces 172 with ridges, bumps, or, inversely, dimples, which facilitate securely holding the needle hub 146 thereby even in an environment in which stray fluids can make holding the needle hub 146 difficult. Together with the textured outer nose-piece surfaces 198 on the sides of the nose piece 178 corresponding with the clip arms 170, the textured outer clip-arm surfaces 172 are configured to form the pair of textured grip pads 182 across the needle hub 146 and the nose piece 178 on opposite sides of the bullet-shaped body of the introducer-needle assembly 140 for holding the RICC insertion assembly 100.

The needle-hub connector 166 includes a needle-hub bore 174 and an optional needle-hub flange 176 about the needle-hub bore 174.

The needle-hub bore 174 of the needle-hub connector 166 is configured to accept a syringe tip (not shown) of the syringe 110 therein for fluidly connecting the introducer needle 104 to the syringe 110. (See FIG. 1 for the fluidly connected introducer needle 104 and syringe 110.) Indeed, the needle-hub bore 174 can have a Luer taper (e.g., a 6% taper) configured to accept the syringe 110 tip therein, which syringe tip can be complementarily configured with a Luer taper.

When present, the needle-hub flange 176 about the needle-hub bore 174 is configured to screw together with internal threads of a threaded collar around the syringe tip of the syringe 110. While the threaded collar of the syringe 110 is also optional, the needle-hub flange 176 advantageously provides a so-called Luer lock-style connection with the internal threads of the threaded collar when both are present. This provides added security against inadvertent disconnection of the introducer needle 104 and the syringe 110 over that provided by an otherwise Luer slip-style connection.

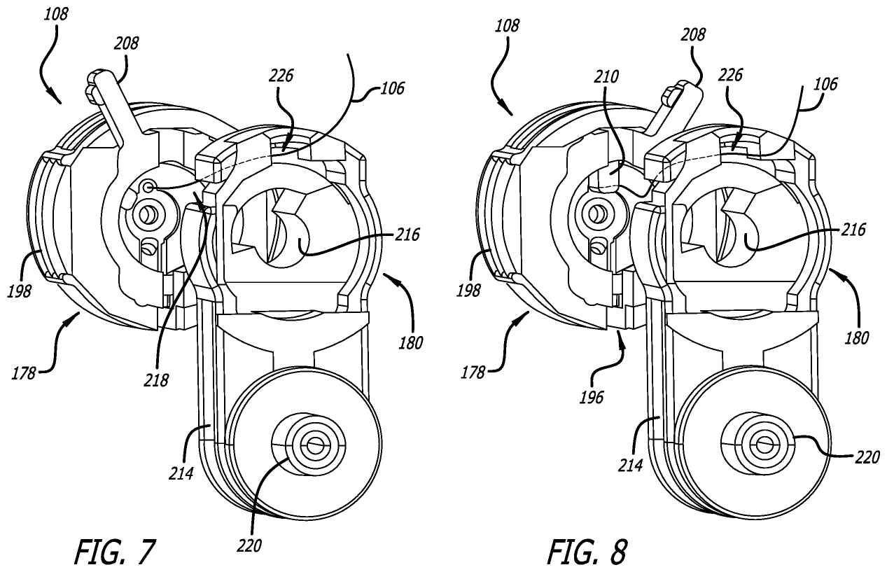

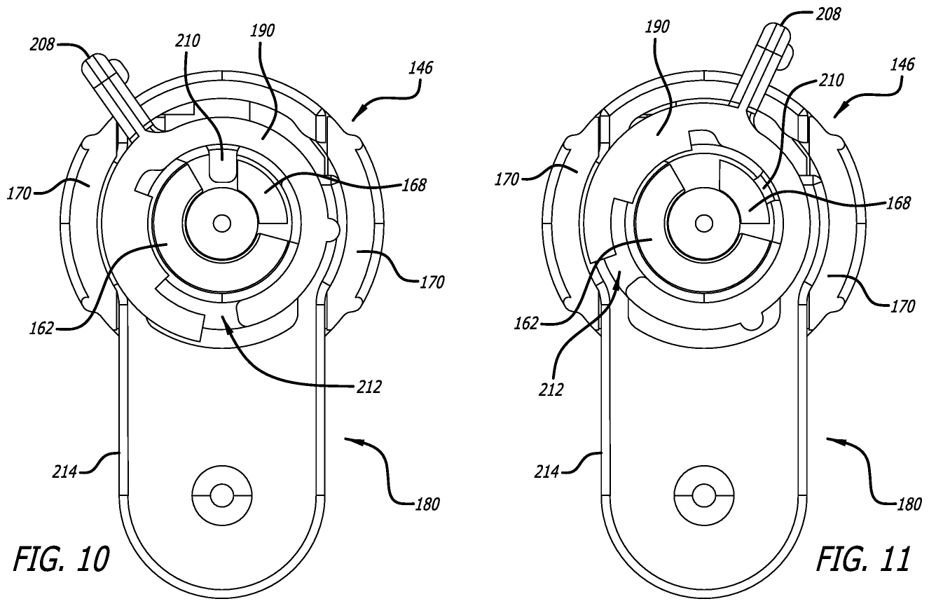

FIGS. 6-8 illustrate various views of the coupler assembly 108 in accordance with some embodiments. While FIG. 9 illustrates a longitudinal cross section of the introducer-needle assembly 140 without the nose-piece housing 184 of the nose piece 178 of the coupler assembly 108, FIG. 9 illustrates another view of at least a portion of the coupler assembly 108. Similarly, FIGS. 10 and 11 illustrate additional views of the coupler assembly 108 by way of the introducer-needle assembly 140 without the nose-piece housing 184 of the nose piece 178, wherein the additional views are from a proximal end of the introducer assembly.

As shown, the coupler assembly 108 includes a nose piece 178 and a tail piece 180 coupled to the nose piece 178. Together, the nose piece 178 and the tail piece 180 of the coupler assembly 108 form a bullet-shaped body configured to be comfortably held overhand in either a left hand for a left-handed venipuncture or a right hand for a right-handed venipuncture with the RICC insertion assembly 100. Notably, the bullet-shaped body is extended with the needle hub 146 of the introducer needle 104 in the introducer-needle assembly 140. Advantageously, the bullet-shaped body of the introducer-needle assembly 140 can include a pair of textured grip pads 182, which facilitate securely holding the introducer-needle assembly 140 thereby even in an environment in which stray fluids can make holding the introducer-needle assembly 140 difficult. Being formed between and the textured outer clip-arm surfaces 172 of the clip arms 170 set forth above and the textured outer nose-piece surfaces 198 of the nose piece 178 set forth below, the pair of textured grip pads 182 extend across the needle hub 146 and the nose piece 178 on opposite sides of the bullet-shaped body of the introducer-needle assembly 140 for holding the RICC insertion assembly 100 during, for example, the left- or right-handed venipuncture.

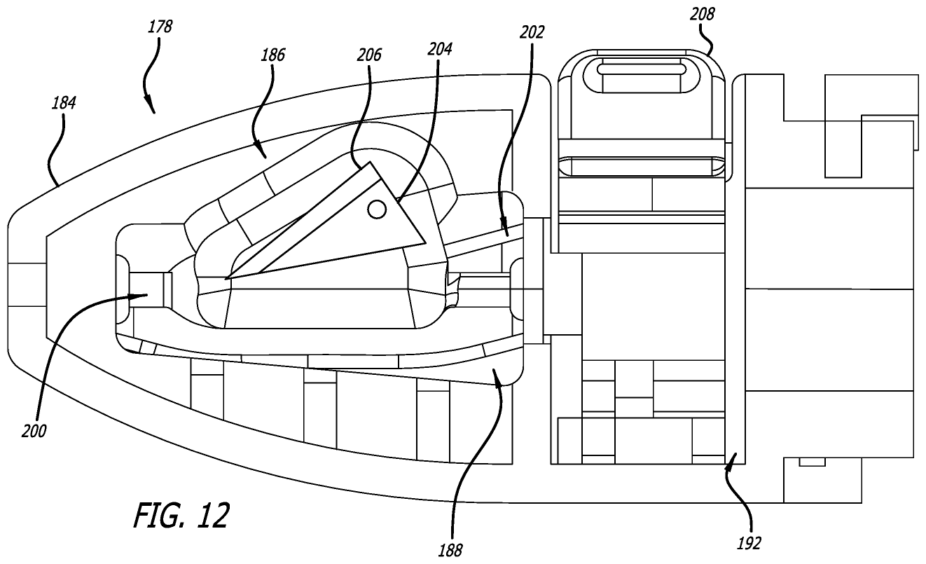

FIG. 12 illustrates a longitudinal cross section of the nose piece 178 of the coupler assembly 108 in accordance with some embodiments.

The nose piece 178 of the coupler assembly 108 includes a nose-piece housing 184, a valve module 186 disposed in a valve-module compartment 188 defined by the nose-piece housing 184, and a lock ring 190 captively but rotatably disposed in a lock-ring compartment 192 defined by the nose-piece housing 184.

The nose-piece housing 184 includes two molded pieces coupled together (e.g., snapped together or fastened or screwed together with screws or bolts) to form at least a distal or tip portion of the foregoing bullet-shaped body of the coupler assembly 108 or introducer-needle assembly 140. An inside of each piece of the two molded pieces includes depressions that define the valve-module compartment 188 and the lock-ring compartment 192 proximal of the valve-module compartment 188 when the two molded pieces are coupled together as shown in FIG. 12. The nose-piece housing 184 of the nose piece 178 also includes a circumferential tab slot 194 and a longitudinal nose piece-housing slot 196 defined between the two molded pieces of the nose-piece housing 184 when the two molded pieces are coupled together. In the introducer-needle assembly 140, the nose piece-housing slot 196 is on a same side as the extension arm 214 of the tail piece 180, thereby opening in an opposite direction to that of the needle slot 150 of the needle shaft 142. Notwithstanding the foregoing, it should be understood the nose piece-housing slot 196 can be alternatively located in another location than that shown. Regardless, the nose piece-housing slot 196 is configured to allow the access guidewire 106 to escape from the nose piece 178 during separation of the nose piece 178 from the access guidewire 106 in the nose piece-separating step of the method set forth below. Advantageously, an outside of each piece of the two molded pieces can include a textured outer nose-piece surface 198 with ridges, bumps, or dimples, which outer nose-piece surfaces 198 facilitate securely holding the nose piece 178 or the coupler assembly 108 thereby even in an environment in which stray fluids can make holding the nose piece 178 or the coupler assembly 108 difficult. Together with the textured outer clip-arm surfaces 172, the textured outer nose-piece surfaces 198 are configured to form the pair of textured grip pads 182 across the nose piece 178 and the needle hub 146 on opposite sides of the bullet-shaped body of the introducer-needle assembly 140 for holding the RICC insertion assembly 100.

The valve module 186 includes an introducer-needle passageway 200, an access-guidewire passageway 202 connected to the introducer-needle passageway 200, and an integrated blade 204 extending into the introducer-needle passageway 200. Notably, the valve module 186 is separable, and the valve-module compartment 188 is further configured with sufficient space to allow the valve module 186 to separate for the escape of the access guidewire 106 from the valve module 186 when the introducer needle 104 is withdrawn from the RICC insertion assembly 100 or the introducer-needle assembly 140 thereof in accordance with the introducer needle-withdrawing step of the method set forth below.

The introducer-needle passageway 200 is configured to allow the introducer needle 104, specifically, an elongate portion of the introducer needle 104 including the sheath 144 over the needle shaft 142, to pass therethrough. In addition, the introducer-needle passageway 200 is configured to seal around the introducer needle 104, specifically, a proximal portion of the elongate portion of the introducer needle 104, in at least the ready-to-deploy state of the RICC insertion assembly 100. Indeed, the valve module 186 is configured to seal over the sheath opening 160 of the sheath 144, which sheath opening 160, as set forth above, opens to the needle slot 150. As further set forth above, the sheath 144 seals the needle slot 150 outside of the valve module 186. Together with the access guidewire 106 sealed in the access-guidewire passageway 202, leak-free aspiration through the introducer needle 104 is enabled for the blood-aspirating step of the method set forth below.

The access-guidewire passageway 202 is configured to allow the access guidewire 106 to pass therethrough and into both the sheath opening 160 of the sheath 144 and the needle channel 154 of the needle shaft 142. Said differently, the access-guidewire passageway 202 is configured to allow the access guidewire 106 to pass therethrough and into the needle lumen 156 of the introducer needle 104 by way of the sheath opening 160 of the sheath 144. Indeed, the access-guidewire passageway 202 connects to the sheath opening 160 of the sheath 144 in at least the ready-to-deploy state of the RICC insertion assembly 100 with the introducer needle 104 within the introducer-needle passageway 200. In addition, the access-guidewire passageway 202 is configured to seal around the access guidewire 106, specifically, the distal portion of the access guidewire 106, in at least the ready-to-deploy state of the RICC insertion assembly 100. Together with the introducer needle 104 sealed in the introducer-needle passageway 200, leak-free aspiration through the introducer needle 104 is enabled for the blood-aspirating step of the method set forth below.

The blade 204 extends from an attachment point in the valve module 186 into the needle slot 150 of the needle shaft 142 such that the blade 204 is disposed in the needle slot 150 under the distal end of the sheath opening 160 of the sheath 144. The blade 204 includes a distal facing blade edge 206 configured to cut the sheath 144 off the needle shaft 142 as the introducer needle 104 is withdrawn in a proximal direction from the coupler assembly 108 in the introducer needle-withdrawing step of the method set forth below. Cutting the sheath 144 off the needle shaft 142 allows the access guidewire 106 to escape from both the needle shaft 142 by way of the needle slot 150 thereof and the coupler assembly 108 by way of the nose piece-housing slot 196 of the nose-piece housing 184.

FIGS. 10 and 11 illustrate views from the proximal end of the introducer-needle assembly 140 with the lock ring 190 to different sides of the nose-piece housing 184 in accordance with some embodiments. Notably, the nose-piece housing 184 and the valve module 186 of the nose piece 178 are removed for expository convenience.

The lock ring 190 includes a lock-ring tab 208 protruding away from a centerline of the lock ring 190, a lock-ring protrusion 210 protruding toward the centerline of the lock ring 190, and a lock-ring gap 212 on an opposite side of the lock ring 190 from the lock-ring protrusion 210, though not necessarily directly opposite the lock-ring protrusion 210. Again, the lock ring 190 is captively but rotatably disposed in the lock-ring compartment 192 defined by the nose-piece housing 184. Notably, the wall of the introducer-needle cradle 216 of the tail piece 180 and the extension-tube protrusion 168 of the needle-hub extension tube 162 extend into the lock-ring compartment 192 of the nose piece 178 in at least the ready-to-deploy state of the RICC insertion assembly 100, thereby providing the two-piece axle of the wall of the introducer-needle cradle 216 and the extension-tube protrusion 168 configured for rotating the lock ring 190 thereupon.

The lock-ring tab 208 extends from the lock ring 190 through the tab slot 194 of the nose-piece housing 184 in which the lock ring 190 is captively disposed. The lock ring 190 is thusly configured to be toggled between a side of the tab slot 194 and an opposite side of the tab slot 194 for restraining the access guidewire 106 from moving in the nose piece 178 and releasing the access guidewire 106 to move in the nose piece 178, respectively. As set forth below, the lock-ring protrusion 210 creates the tortured guidewire passageway that restrains the access guidewire 106 from moving in the nose piece 178 when the lock-ring tab 208 is to the side of the tab slot 194 of the nose-piece housing 184. In addition, the lock-ring protrusion 210 creates the open guidewire passageway that allows the access guidewire 106 to move in the nose piece 178 when the lock-ring tab 208 is to the opposite side of the tab slot 194 of the nose-piece housing 184.

The lock-ring protrusion 210 is positioned on the lock ring 190 to create a tortured guidewire passageway therearound for restraining the access guidewire 106 from moving in the nose piece 178 when the lock-ring tab 208 is to the side of the tab slot 194 of the nose-piece housing 184. In the introducer-needle assembly 140, the tortured guidewire passageway extends from the access-guidewire passageway 202 of the valve module 186, around the lock-ring protrusion 210, over the needle-hub extension tube 162 through a remainder of the nose piece 178 and the tail piece 180, and out the guidewire through hole 226 formed between the tail piece 180 and the needle hub 146 of the introducer needle 104. The lock-ring protrusion 210 is further positioned on the lock ring 190 to create an open guidewire passageway for allowing the access guidewire 106 to move in the nose piece 178 when the lock-ring tab 208 is to the opposite side of the tab slot 194 of the nose-piece housing 184. Notably, a length of the lock-ring protrusion 210 is short of an entire width of the lock ring 190 such that the lock-ring protrusion 210 can be proximal or distal of the extension-tube protrusion 168 of the needle-hub extension tube 162 in at least the ready-to-deploy state of the RICC insertion assembly 100. (See FIG. 9, where the lock-ring protrusion 210 is positioned on the lock ring 190 such that it is proximal of the extension-tube protrusion 168.) The lock-ring protrusion 210 being proximal or distal of the extension-tube protrusion 168 obviates any interference of the extension-tube protrusion 168 with the lock-ring protrusion 210 when the lock ring 190 is rotated in the lock-ring compartment 192 over the two-piece axle formed by the wall of the introducer-needle cradle 216 of the tail piece 180 and the extension-tube protrusion 168 of the needle-hub extension tube 162.

The lock-ring gap 212 being on the opposite side of the lock ring 190 from the lock-ring protrusion 210 enables the access guidewire 106 to escape from the lock ring 190 when separating the nose piece 178 from the access guidewire 106 in accordance with the nose piece-separating step of the method set forth below. Advantageously, the lock ring 190 can be configured such that the lock-ring gap 212 aligns with the nose piece-housing slot 196 of the nose-piece housing 184 when the lock-ring tab 208 is to the opposite side of the tab slot 194 creating the open guidewire passageway, thereby releasing the access guidewire 106 to move in the nose piece 178 for escape from the nose piece 178 through both the lock-ring gap 212 and the nose piece-housing slot 196 in the nose piece-separating step of the method set forth below.

The tail piece 180 includes an extension arm 214, an introducer-needle cradle 216, and a longitudinal tail-piece slot 218.

The extension arm 214 includes an extension-arm connector 220 and a guidewire-holding point 222 by which the proximal end of the access guidewire 106 is held in at least the ready-to-deploy state of the RICC insertion assembly 100. The extension arm 214 terminates with the extension-arm connector 220, which includes a male Luer connector configured to connect with a female counterpart thereof such as a Luer connector of the RICC 102. Indeed, as shown in FIG. 1, the extension-arm connector 220 is connected with a Luer connector of the RICC 102. The guidewire-holding point 222 can be opposite the extension-arm connector 220 such as distal of the extension-arm connector 220, as shown in FIG. 9, with an access-guidewire passageway 224 passing through a center of each of the extension-arm connector 220 and the guidewire-holding point 222 therebetween. The proximal portion of the access guidewire 106 is threaded through the access-guidewire passageway 224 so that the access guidewire 106 extends from the extension-arm connector 220, into the Luer connector of the RICC 102, and along, for example, the primary lumen 128 of the RICC 102 in at least the ready-to-deploy state of the RICC insertion assembly 100. The proximal end of the access guidewire 106 can be adhered to the guidewire-holding point 222 to stop the proximal end of the access guidewire 106 from passing through the access-guidewire passageway 224, thereby obviating inadvertent over advancement of the access guidewire 106 in a distal direction. That said, the proximal end of the access guidewire 106 can alternatively or additionally include a stop (e.g., a hub, a ball, a stopper knot, etc.) configured to stop the proximal end of the access guidewire 106 from passing through the access-guidewire passageway 224, thereby also obviating inadvertent over advancement of the access guidewire 106 in the distal direction.

The extension arm 214 can be molded with a remainder of the tail piece 180 such that the extension arm 214 is integral therewith. Alternatively, the extension arm 214 is separately molded and connected to the tail piece 180. Regardless, the extension arm 214 is fixedly and immovably connected to the same side of the coupler assembly 108 or the introducer-needle assembly 140 including the nose piece-housing slot 196. In view of the extension arm 214 being connected to the same side of the coupler assembly 108 or the introducer-needle assembly 140 as the nose piece-housing slot 196, each of the RICC insertion assembly 100 and the foregoing subassemblies have an immediately recognizable orientation.

The introducer-needle cradle 216 is configured to cradle the needle-hub extension tube 162 distally extending from the needle hub 146 and form the two-piece axle therewith in at least a ready-to-deploy state of the RICC insertion assembly 100. Indeed, the introducer-needle cradle 216 or a wall thereof approximates a longitudinal cross-section of a tube or pipe distally extending from the tail piece 180 for cradling the needle-hub extension tube 162. The wall of the introducer-needle cradle 216 is at least partially complemented by the extension-tube protrusion 168 in the distal portion the needle-hub extension tube 162 to form the two-piece axle in the lock-ring compartment 192 for rotating the lock ring 190 thereupon.

The tail-piece slot 218 is configured to allow the access guidewire 106 to escape from the tail piece 180 through the tail-piece slot 218 subsequent to both withdrawal of the introducer needle 104 from the coupler assembly 108 and separation of the tail piece 180 from the coupler assembly 108. A width of the tail-piece slot 218 is, thusly, sized in accordance with the diameter of the access guidewire 106. Notably, the introducer-needle cradle 216 opens toward the tail-piece slot 218, thereby facilitating the escape of the guidewire from the tail piece 180 subsequent to the withdrawal of the introducer needle 104 from the coupler assembly 108 and separation of the tail piece 180 from the coupler assembly 108.

The tail piece 180 can also include a whole or partial guidewire through hole 226 therethrough such as that shown in FIG. 9 between the tail piece 180 and the needle hub 146. Such a guidewire through hole, which is sized in accordance with the diameter of the access guidewire 106, is configured to allow the access guidewire 106 to pass into the coupler assembly 108 or the introducer-needle assembly 140.

FIG. 1 illustrates the access guidewire 106 as part of the RICC insertion assembly 100 in accordance with some embodiments.

The access guidewire 106 includes a proximal portion including a proximal end and a distal portion including a distal end. In at least the ready-to-deploy state of the RICC insertion assembly 100, the proximal end of the access guidewire 106 is held by the guidewire-holding point 222 of the extension arm 214. The proximal portion of the access guidewire 106 extends from the guidewire-holding point 222 of the extension arm 214, through the access-guidewire passageway 224, through the extension-arm connector 220, into the Luer connector of the RICC 102, and along the primary lumen 128 of the RICC 102. The distal portion of the access guidewire 106 also extends along the primary lumen 128 of the RICC 102, but the distal portion of the access guidewire 106 further extends out the distal end of the RICC 102, through the guidewire through hole 226 through the tail piece 180, over the needle-hub extension tube 162 through a remainder of the tail piece 180 and the nose piece 178, through the lock ring 190 in the lock-ring compartment 192, into the valve module 186 by way of the access-guidewire passageway 202, into the needle lumen 156 of the introducer needle 104 through the sheath opening 160 of the sheath 144 and the needle slot 150 of the needle shaft 142, and along the needle lumen 156 of the introducer needle 104 in the ready-to-deploy state of the RICC insertion assembly 100. As shown in FIG. 1, the distal end of the access guidewire 106 is disposed in the needle lumen 156 of the introducer needle 104 just proximal of the needle tip 148 in at least the ready-to-deploy state of the RICC insertion assembly 100. Again, the proximal and distal ends of the access guidewire 106 enforce the loop in the access guidewire 106 in at least the ready-to-deploy state of the RICC insertion assembly 100, which loop the RICC 102 is disposed over, thereby keeping the RICC insertion assembly 100 in a relatively compact form.

The access guidewire 106 can include a guidewire tip 228 in the distal portion of the access guidewire 106, which can adopt a ‘J’ shape configured to prevent puncturing a back wall of a blood vessel. Such a guidewire tip assumes a straightened state in at least the ready-to-deploy state of the RICC insertion assembly 100 and a curved state when the guidewire tip 228 is advanced beyond the needle tip 148 (e.g., advanced into a blood-vessel lumen) in a deployed state of the RICC insertion assembly 100.

The access guidewire 106 can further include a bare-wire portion and a wound-wire portion proximal of the bare-wire portion. While not shown, the bare-wire portion, when present, distally extends through the access-guidewire passageway 202 of the valve module 186 in at least the ready-to-deploy state of the RICC insertion assembly 100 such that the valve module 186 forms a fluid-tight seal around the bare-wire portion of the access guidewire 106. Notably, the foregoing bare-wire portion can instead be a flat-wound or ground-wound portion of the access guidewire 106, wherein the flat-wound portion includes windings of a tape instead of a round wire, and wherein the ground-wound portion includes windings of a round wire ground down to flatten the windings.

### Methods

Methods include a method for inserting the RICC 102 into a blood-vessel lumen of a patient. Such a method includes one or more steps selected from a RICC insertion assembly-obtaining step, a needle tract-establishing step, a blood-aspirating step, an access guidewire-advancing step, an access guidewire-restraining step, an introducer needle-withdrawing step, a tail piece-separating step, an access guidewire-releasing step, a nose piece-separating step, a RICC-advancing step, an access guidewire-withdrawing step, a maneuver guidewire-advancing step, another RICC-advancing step, and a maneuver guidewire-withdrawing step.

The RICC insertion assembly-obtaining step includes obtaining the RICC insertion assembly 100. As set forth above, the RICC insertion assembly 100 includes the RICC 102, the introducer needle 104 including the sheath 144 over the needle shaft 142, and the access guidewire 106 coupled together by the coupler assembly 108. The proximal end of the access guidewire 106 is held by the extension arm 214 of the tail piece 180 of the coupler assembly 108. In addition, the distal end of the access guidewire 106 is disposed in the introducer needle 104 by way of the valve module 186 disposed in the nose-piece housing 184 of the nose piece 178 of the coupler assembly 108 such that the proximal and distal ends of the access guidewire 106 enforce the loop in the access guidewire 106. The RICC 102 is disposed over the access guidewire 106 in at least the ready-to-deploy state of the RICC insertion assembly 100, which keeps the RICC insertion assembly 100 in a relatively compact form.

The needle tract-establishing step includes establishing a needle tract from an area of skin to the blood-vessel lumen with the introducer needle 104. The needle tract-establishing step can also include ensuring blood flashback while establishing the needle tract. Ensuring blood flashback while establishing the needle tract includes ensuring blood flashes back into the needle hub 146 of the introducer needle 104, particularly when the needle hub 146 is clear and colorless, the syringe tip of the syringe 110 fluidly connected to the introducer needle 104, a barrel of the syringe 110, or a combination thereof. A slight vacuum can be drawn with the syringe 110 while establishing the needle tract such that the blood flashes back into at least the needle hub 146 of the introducer needle 104 upon establishing the needle tract. Ensuring the blood flashes back in accordance with the foregoing confirms the needle tract extends into the blood-vessel lumen.

The blood-aspirating step includes aspirating blood with the syringe 110 coupled to the needle hub 146 of the introducer needle 104 for confirmation the needle tract extends into the blood-vessel lumen, notably before the withdrawing of the introducer needle 104 from the RICC insertion assembly 100 or the introducer-needle assembly 140 thereof. Again, the sheath 144 over the needle shaft 142 seals the needle slot 150 of the needle shaft 142 thereunder. In particular, the sheath 144 seals the needle slot 150 outside of the valve module 186. The valve module 186, in turn, seals over the sheath opening 160 of the sheath 144, which sheath opening 160 allows the access guidewire 106 to pass into the needle lumen 156 of the introducer needle 104 in the ready-to-deploy state of the RICC insertion assembly 100. The valve module 186 also seals around the distal portion of the access guidewire 106. Such seals enable the syringe 110 to aspirate blood in the blood-aspirating step.

The access guidewire-advancing step includes advancing the distal end of the access guidewire 106 into the blood-vessel lumen from its initial location in the elongate portion of the introducer needle 104 or needle shaft 142 thereof just proximal of the needle tip 148, thereby securing blood-vessel access for the advancing of the catheter tube 112 of the RICC 102 into the blood-vessel lumen.

The access guidewire-restraining step includes restraining the access guidewire 106 from moving in the nose piece 178 of the coupler assembly 108 after the advancing of the access guidewire 106 into the blood-vessel lumen so as to not withdraw the access guidewire 106 from the blood-vessel lumen during the withdrawing of the introducer needle 104 from the RICC insertion assembly 100 or the introducer-needle assembly 140 thereof or the separating of the tail piece 180 from the nose piece 178 of the coupler assembly 108. The restraining of the access guidewire 106 from moving in the nose piece 178 includes rotating the lock ring 190, which lock ring 190 has the lock-ring protrusion 210 that protrudes toward the centerline of the lock ring 190 as set forth above. The rotating of the lock ring 190 includes pushing the lock-ring tab 208 of the lock ring 190 to the side or the opposite side of the tab slot 194 of the nose-piece housing 184 through which tab slot 194 the lock-ring tab 208 extends. Notably, the rotating of the lock ring 190 is over the two-piece axle formed between the wall of the introducer-needle cradle 216 and the extension-tube protrusion 168 of the needle-hub extension tube 162 extending into the lock-ring compartment 192 including the lock ring 190. The rotating of the lock ring 190 in the access guidewire-restraining step creates the tortured guidewire passageway around the lock-ring protrusion 210 for the restraining of the access guidewire 106 from moving in the nose piece 178 of the coupler assembly 108.

The introducer needle-withdrawing step includes withdrawing the introducer needle 104 by the needle hub 146 from the RICC insertion assembly 100 or the introducer-needle assembly 140 thereof leaving the access guidewire 106 in place in the blood-vessel lumen. The introducer needle-withdrawing step includes simultaneously cutting the sheath 144 off the needle shaft 142 with the blade 204 of the valve module 186 disposed in the valve-module compartment 188 defined by the nose-piece housing 184 of the nose piece 178 when the introducer needle 104 is withdrawn from the RICC insertion assembly 100 or the introducer-needle assembly 140 thereof. The cutting of the sheath 144 off the needle shaft 142 allows the access guidewire 106 to escape from the needle shaft 142 by way of the needle slot 150. Again, the needle shaft 142 includes the needle slot 150 extending from the proximal portion of the needle shaft 142 through the needle tip 148, which needle slot 150 allows the access guidewire 106 to escape from the needle shaft 142 with the withdrawing of the introducer needle 104 from the RICC insertion assembly 100 or the introducer-needle assembly 140 thereof. Notably, the valve module 186 around the elongate portion of the introducer needle 104 separates to allow the access guidewire 106 to further escape from the valve module 186 when the introducer needle 104 is withdrawn from the RICC insertion assembly 100 or the introducer-needle assembly 140 thereof.

The tail piece-separating step includes separating the tail piece 180 from the nose piece 178 of the coupler assembly 108. Again, the tail piece 180 includes the longitudinal tail-piece slot 218 that allows the access guidewire 106 to escape from the tail piece 180 subsequent to the withdrawal of the introducer needle 104 from the RICC insertion assembly 100 or the introducer-needle assembly 140 thereof. The tail piece 180 becomes a handle for the access guidewire 106 after the separating of the tail piece 180 from the nose piece 178 of the coupler assembly 108.

The access guidewire-releasing step includes releasing the access guidewire 106 to move in the nose piece 178 of the coupler assembly 108 after the withdrawing of the introducer needle 104 from the RICC insertion assembly 100 or the introducer-needle assembly 140 thereof as well as the separating of the tail piece 180 from the nose piece 178 of the coupler assembly 108. The releasing of the access guidewire 106 to move in the nose piece 178 includes rotating the lock ring 190 opposite to that for the restraining of the access guidewire 106 from moving in the nose piece 178 of the coupler assembly 108. The rotating of the lock ring 190 in the access guidewire-releasing step creates the open guidewire passageway for the releasing of the access guidewire 106 to move in the nose piece 178.

The nose piece-separating step includes separating the nose piece 178 from the access guidewire 106 after the releasing of the access guidewire 106 to move in the nose piece 178. The lock ring 190 includes the lock-ring gap 212 on the opposite side of the lock ring 190 from the lock-ring protrusion 210. The lock-ring gap 212 allows the access guidewire 106 to escape from the lock ring 190 during the separating of the nose piece 178 from the access guidewire 106. In addition, the nose-piece housing 184 of the nose piece 178 includes the nose piece-housing slot 196 on the same side of the coupler assembly 108 as the extension arm 214 of the tail piece 180 of the coupler assembly 108. The nose piece-housing slot 196 also allows the access guidewire 106 to escape from the nose piece 178 during the separating of the nose piece 178 from the access guidewire 106.

The RICC-advancing step includes advancing the catheter tube 112 of the RICC 102 over the access guidewire 106 and into the blood-vessel lumen, thereby inserting the RICC 102 into the blood-vessel lumen.

The access guidewire-withdrawing step includes withdrawing the access guidewire 106 from the blood-vessel lumen after the inserting of the RICC 102 into the blood-vessel lumen. The withdrawing of the access guidewire 106 from the blood-vessel lumen also includes removing the Luer connector of the RICC 102 from the extension-arm connector 220 of the extension arm 214, thereby decoupling the RICC 102 from the tail piece 180.

The maneuver guidewire-advancing step includes advancing a maneuver guidewire into the blood-vessel lumen by way of the primary lumen 128 of the RICC 102 and to a lower ⅓ of a superior vena cava (“SVC”) of a heart of the patient.

The other RICC-advancing step includes advancing the distal portion of the catheter tube 112 farther into the blood-vessel lumen over the maneuver guidewire to the lower ⅓ of the SVC of the heart of the patient.

The maneuver guidewire-withdrawing step includes withdrawing the maneuver guidewire leaving the catheter tube 112 in place in the lower ⅓ of the SVC.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A rapidly insertable central catheter (“RICC”) insertion assembly, comprising:
a RICC;
an introducer needle including:
a needle shaft including a longitudinal needle slot extending from a proximal portion of the needle shaft through a distal needle tip;
a sheath over the needle shaft sealing the needle slot thereunder except for that under a sheath opening in a proximal portion of the sheath; and
a needle hub over the proximal portions of the needle shaft and the sheath, the needle hub;
an access guidewire including:
a proximal portion including a proximal end; and
a distal portion including a distal end disposed in the introducer needle just proximal of the needle tip in at least a ready-to-deploy state of the RICC insertion assembly; and
a coupler assembly coupling the RICC, the introducer needle, and the access guidewire together, the coupler assembly including:
a nose piece; and
a tail piece coupled to the nose piece, the tail piece of the coupler assembly including an extension arm by which the proximal end of the access guidewire is held, the proximal and distal ends of the access guidewire enforcing a loop in the access guidewire over which the RICC is disposed in at least the ready-to-deploy state of the RICC insertion assembly.

2. The RICC insertion assembly according to claim 1, wherein the nose piece of the coupler assembly includes a nose-piece housing defining a valve-module compartment and a lock-ring compartment proximal of the valve-module compartment.

3. The RICC insertion assembly according to claim 2, wherein the nose piece of the coupler assembly further includes a valve module disposed in the valve-module compartment, the valve module including an introducer-needle passageway and an access-guidewire passageway connecting thereto, within which passageways the introducer needle and the distal portion of the access guidewire are correspondingly sealed in at least the ready-to-deploy state of the RICC insertion assembly, thereby enabling leak-free aspiration through the introducer needle.

4. The RICC insertion assembly according to claim 3, wherein the sheath opening of the sheath opens toward an opposite side of the coupler assembly than that including the extension arm, the access-guidewire passageway connecting to the sheath opening in at least the ready-to-deploy state of the RICC insertion assembly with the introducer needle within the introducer-needle passageway.

5. The RICC insertion assembly according to claim 3, wherein the valve module includes an integrated blade disposed in the needle slot under a distal end of the sheath opening, the blade including a distal-facing blade edge configured to cut the sheath off the needle shaft during withdrawal of the introducer needle from the coupler assembly by the needle hub, thereby allowing the access guidewire to escape from the needle shaft by way of the needle slot thereof.

6. The RICC insertion assembly according to claim 2, wherein the nose piece of the coupler assembly further includes a lock ring captively but rotatably disposed in the lock-ring compartment.

7. The RICC insertion assembly according to claim 6, wherein the tail piece of the coupler assembly includes an introducer-needle cradle distally extending from the tail piece, the introducer-needle cradle configured to cradle a needle-hub extension tube distally extending from the needle hub in at least a ready-to-deploy state of the RICC insertion assembly.

8. The RICC insertion assembly according to claim 7, wherein a wall of the introducer-needle cradle approximates a longitudinal cross-section of a pipe, the needle-hub extension tube including an arcuate extension-tube protrusion in a distal portion thereof configured to at least partially complement the wall of the introducer-needle cradle, thereby forming a two-piece axle configured for rotating the lock ring thereupon.

9. The RICC insertion assembly according to claim 7, wherein the tail piece of the coupler assembly includes a longitudinal tail-piece slot to which the introducer-needle cradle opens, the tail-piece slot configured to allow the access guidewire to escape from the tail piece subsequent to withdrawal of the introducer needle from the coupler assembly and separation of the tail piece from the coupler assembly.

10. The RICC insertion assembly according to claim 7, wherein the lock ring includes a lock-ring protrusion protruding toward a centerline of the lock ring, the lock-ring protrusion positioned on the lock ring to create a tortured guidewire passageway therearound for restraining the access guidewire from moving when a lock-ring tab extending away from the centerline of the lock ring is to a side of a tab slot of the nose-piece housing through which tab slot the lock-ring tab extends.

11. The RICC insertion assembly according to claim 10, wherein the lock-ring protrusion is further positioned on the lock ring to create an open guidewire passageway for the access guidewire when the lock-ring tab is to an opposite side of the tab slot of the nose-piece housing of the coupler assembly.

12. The RICC insertion assembly according to claim 10, wherein the lock ring includes a lock-ring gap on an opposite side of the lock ring from the lock-ring protrusion, the lock-ring gap configured to allow the access guidewire to escape from the lock ring during separation of the nose piece from the access guidewire.

13. The RICC insertion assembly according to claim 2, wherein the nose-piece housing of the nose piece includes a longitudinal nose piece-housing slot on a same side of the coupler assembly as the extension arm of the tail piece, the nose piece-housing slot configured to allow the access guidewire to escape from the nose piece during separation of the nose piece from the access guidewire.

14. The RICC insertion assembly according to claim 1, wherein the needle hub of the introducer needle includes a pair of clip arms distally extending from opposite sides of the needle hub, the clip arms configured to extend completely over corresponding sides of the tail piece of the coupler assembly to at least the nose piece thereof in at least a ready-to-deploy state of the RICC insertion assembly.

15. The RICC insertion assembly according to claim 14, wherein the clip arms include textured outer clip-arm surfaces and the nose piece of the coupler assembly includes textured outer nose-piece surfaces on sides of the nose piece corresponding with the clip arms, the outer clip-arm surfaces and the outer nose-piece surfaces configured to form a pair of textured grip pads on opposite sides of the RICC insertion assembly across the nose piece and the needle hub.

16. The RICC insertion assembly according to claim 1, wherein the extension arm terminates with an extension-arm connector connected to a Luer connector of the RICC in at least the ready-to-deploy state of the RICC insertion assembly.

17. The RICC insertion assembly according to claim 16, wherein the extension-arm connector includes a male Luer connector, the Luer connector of the RICC a female counterpart to the male Luer connector of the extension-arm connector.

18. The RICC insertion assembly according to claim 1, further comprising a syringe fluidly coupled to the introducer needle in at least the ready-to-deploy state of the RICC insertion assembly.

19. A method for inserting a rapidly insertable central catheter (“RICC”) into a blood-vessel lumen of a patient, comprising:
obtaining a RICC insertion assembly including the RICC, an introducer needle including a sheath over a needle shaft, and an access guidewire coupled together by a coupler assembly, a proximal end of the access guidewire held by an extension arm of a tail piece of the coupler assembly and a distal end of the access guidewire disposed in the introducer needle by way of a valve module disposed in a nose-piece housing of a nose piece of the coupler assembly such that the proximal and distal ends of the access guidewire enforce a loop in the access guidewire, over which the RICC is disposed in at least a ready-to-deploy state of the RICC insertion assembly;
establishing a needle tract from an area of skin to the blood-vessel lumen with the introducer needle;
advancing the distal end of the access guidewire from its initial location in the needle shaft just proximal of a needle tip of the needle shaft into the blood-vessel lumen;
withdrawing the introducer needle by a needle hub thereof from the coupler assembly leaving the access guidewire in place in the blood-vessel lumen, the needle shaft including a longitudinal needle slot extending from a proximal portion of the needle shaft through the needle tip allowing the access guidewire to escape therefrom with the withdrawing of the introducer needle from the coupler;
separating the tail piece from the nose piece of the coupler assembly, the tail piece including a longitudinal tail-piece slot allowing the access guidewire to escape therefrom subsequent to the withdrawal of the introducer needle from the coupler assembly; and
advancing a catheter tube of the RICC over the access guidewire for the inserting of the RICC into the blood-vessel lumen.

20. The method according to claim 19, further comprising:
aspirating blood with a syringe coupled to the needle hub of the introducer needle for confirmation the needle tract extends into the blood-vessel lumen before the withdrawing of the introducer needle from the coupler assembly, the sheath over the needle shaft sealing the needle slot thereunder for the aspirating of the blood with the syringe.

21. The method according to claim 19, wherein the withdrawing of the introducer needle from the coupler assembly includes simultaneously cutting the sheath off the needle shaft with an integrated blade of a valve module disposed in a valve-module compartment defined by the nose-piece housing of the nose piece, the cutting of the sheath off the needle shaft allowing the access guidewire to escape from the needle shaft by way of the needle slot thereof.

22. The method according to claim 19, further comprising restraining the access guidewire from moving in the RICC insertion assembly after the advancing of the access guidewire into the blood-vessel lumen so as to not withdraw the access guidewire from the blood-vessel lumen during the withdrawing of the introducer needle from the coupler assembly or the separating of the tail piece from the nose piece of the coupler assembly, the restraining of the access guidewire from moving in the RICC insertion assembly including rotating a lock ring having a lock-ring protrusion protruding toward a centerline of the lock ring, thereby creating a tortured guidewire passageway around the lock-ring protrusion for the restraining of the access guidewire from moving in the RICC insertion assembly.

23. The method according to claim 22, wherein the rotating of the lock ring includes pushing a lock-ring tab of the lock ring to a side or an opposite side of a tab slot of the nose-piece housing through which tab slot the lock-ring tab extends.

24. The method according to claim 22, wherein the rotating of the lock ring is over a two-piece axle formed between a wall of an introducer-needle cradle distally extending from the tail piece of the coupler assembly and an arcuate extension-tube protrusion of a needle-hub extension tube distally extending from the needle hub of the introducer needle within a lock-ring compartment defined by the nose-piece housing of the nose piece in which the lock ring is captively disposed.

25. The method according to claim 22, further comprising releasing the access guidewire to move in the nose piece after the withdrawing of the introducer needle from the coupler assembly and the separating of the tail piece from the nose piece of the coupler assembly, the releasing of the access guidewire to move in the nose piece including rotating the lock ring opposite to that for the restraining of the access guidewire from moving in the RICC insertion assembly, thereby creating an open guidewire passageway for the releasing of the access guidewire to move in the nose piece.

26. The method according to claim 25, further comprising separating the nose piece from the access guidewire after the releasing of the access guidewire to move in the nose piece, the lock ring including a lock-ring gap on an opposite side of the lock ring from the lock-ring protrusion, thereby allowing the access guidewire to escape from the lock ring during the separating of the nose piece from the access guidewire.

27. The method according to claim 24, wherein the nose-piece housing of the nose piece includes a longitudinal nose piece-housing slot on a same side of the coupler assembly as the extension arm of the tail piece of the coupler assembly allowing the access guidewire to escape from the nose piece during the separating of the nose piece from the access guidewire.

28. The method according to claim 26, wherein the tail piece becomes a handle for the access guidewire after the separating of the tail piece from the nose piece of the coupler assembly.

29. The method according to claim 19, further comprising withdrawing the access guidewire from the blood-vessel lumen after the inserting of the RICC into the blood-vessel lumen, the withdrawing of the access guidewire from the blood-vessel lumen including removing a Luer connector of the RICC from an extension-arm connector of the extension arm, thereby decoupling the RICC from the tail piece.
